(19) United States
(12) Patent Application Publication
(10) Pub. No.: US 2021/0290720 A1
(43) Pub. Date: Sep. 23, 2021

(54) EXTRACTS OF CITRUS AURANTIUM VAR. BERGAMIA, COMBINATIONS THEREOF AND FORMULATIONS CONTAINING THEM

(71) Applicant: HERBAL E ANTIOXIDANT DERIVATIVES S.R.L. ED IN FORMA ABBREVIATA H&AD S.R.L., Bianco (RC) (IT)

(72) Inventors: Ezio Bombardelli, Gropello Cairoli (PV) (IT); Elena Lombardo, Bianco (RC) (IT); Giuseppe Trunfio, Torregrotta (ME) (IT)

(73) Assignee: HERBAL E ANTIOXIDANT DERIVATIVES S.R.L. ED IN FORMA ABBREVIATA H&AD S.R.L., Bianco (RC) (IT)

(21) Appl. No.: 17/262,514

(22) PCT Filed: Jul. 22, 2019
(86) PCT No.: PCT/IB2019/056229

§ 371 Date: Jan. 22, 2021

(30) Foreign Application Priority Data
Jul. 23, 2018 (IT) 102018000007433

(51) Int. Cl. A61K 36/752; A61K 9/48

(52) U.S. Cl. CPC A61K 36/752; A61K 9/4858; A61K 9/4825; A61K 2236/39; A61K 2236/333; A61K 2236/51; A61K 2236/53; A61K 2236/15

(57) ABSTRACT

The present invention relates to a novel extract of Citrus aurantium var. bergamia obtainable from the aerial parts of the plant, in particular the leaves, branches and bark. The extract according to the invention has a content of neoeriocitrin and oligomeric catechin procyanidins greater than that of the known extracts obtained from the fruit. The extract is useful for the prevention and treatment of dyslipidaemia, hyperglycaemia, vascular inflammation and hepatic steatosis, optionally in combination with other extracts.

The present invention relates to a novel extract of Citrus aurantium var. bergamia obtainable from the aerial parts of the plant, in particular the leaves, branches and bark. The extract according to the invention has a content of neoeriocitrin and oligomeric catechin procyanidins greater than that of the known extracts obtained from the fruit. The extract is useful for the prevention and treatment of dyslipidaemia, hyperglycaemia, vascular inflammation and hepatic steatosis, optionally in combination with other extracts.

### PRIOR ART

Extracts of bergamot orange (Citrus aurantium var. bergamia), a plant which is only present on the Ionian coast of Calabria (Italy), are studied for their beneficial activities, especially their lipid- and cholesterol-reducing activity.

U.S. Pat. No. 8,741,362 describes fresh bergamot orange fruit extracts characterised by a content of the flavonoids neoeriocitrin, naringin and neohesperidin amounting to 29.6±6.0, 32.4±4.0 and 38.0±6% respectively (determined by the HPLC method). The mixture of flavonoids constitutes about 40% by weight of the extract, and can reach 50%, depending on the harvesting period and the ripeness of the fruit. Other flavonoids are also present in lower amounts, such as melitidin and brutieridin (derivatives of eriocitrin and neoeriocitrin), luteolin derivatives and oligomeric polymers thereof. Bergamot orange extracts are widely used as nutraceuticals. The limited availability of the fruit, due to its limited production area, constitutes an obstacle to increasing its industrial exploitation.

There is consequently a need to find alternative sources of bergamot orange fruit for the production of extracts which have pharmacological properties equal to or better than those of the conventional fruit extracts, but with almost unlimited, or in any event much wider, availability of plant material for extraction.

### DESCRIPTION OF THE INVENTION

It has now been discovered that the aerial parts, in particular the leaves, of Citrus aurantium var. bergamia or Citrus aurantium var. myrtifolia can be used as an alternative to the fruit to prepare extracts useful as active ingredients of formulations for the control of lipid metabolism and treatment or prevention of metabolic syndrome.

The subject of the invention is therefore extracts of the aerial parts, especially the leaves, of bergamot orange.

In particular the leaves of the femminello and troyan cultivars of Citrus aurantium var. bergamia have proved to represent an advantageous alternative source to the bergamot orange fruit previously used, with consequent advantages associated with the availability of annual, not just seasonal material.

The extracts according to the invention are characterised by a preponderant neoeriocitrin content, 50 to 90% greater than that of the other flavonoids, an oligomeric procyanidin content ranging from 15 to 30% by weight of the extract, but standardisable to about 20%, and the absence of furanocoumarins and coumarins, which are usually present in the fruit.

The absence of furanocoumarins and coumarins is advantageous because it eliminates the risk of allergic or adverse reactions relating to blood coagulation and the blood count, which are significant risks, especially in chronic treatments, as in the case of atherosclerotic and familial hyperlipidaemia.

The oligomeric procyanidin part was evaluated by quantitation with SEC-Chromatography, as reported below. The resulting product has an HPLC-assayable flavonoid content ranging between 35 and 65%, normally 40%, and a total flavonoid content of 85%, determined by the Folin Ciocalteu colorimetric method (Int. J. Food Sci. Technol., 37 (2002), pp. 153-161; Evid Based Complement Alternat Med. 2015; 2015: 957031. Briefly, 50 μL of methanol/water solutions of the samples was added to 450 μL of deionised water, 500 μL of Folin-Ciocalteu reagent (aqueous solution of phosphomolybdate and phosphotungstate) and 500 μL of 10% sodium carbonate solution; after incubation in the dark for 1 h at room temperature, the absorbance was read off at 786 nm against a blank containing 50 μL of the same solvent. The total phenol content is expressed in mg of gallic acid equivalents (GAE/g of dried extract). The flavonoids were assayed by the HPLC method on a Kinetex 5 μC18 100 A (150×4.6) mm column with a gradient of acetonitrile (eluent A) and an 0.088% solution of acetic acid in water (v:v) (eluent B), injection volume 3.0 μL (methanol:water 1:1), temperature 30°±1° C. The elution gradient is shown in the table 1.

| | | | Flow rate | Eluent | Eluent |
|---|---|---|---|---|---|
| Step | | time (min) | (mL/min) | A (%) | B (%) |
| 0 | Equilibration | 6.0 | 1.4 | 7.0 | 93.0 |
| 1 | Run | 2.0 | 1.4 | 19.0 | 81.0 |
| 2 | Run | 8.0 | 1.4 | 20.4 | 79.6 |
| 3 | Run | 3.0 | 1.4 | 60.0 | 40.0 |
| 4 | Run | 1.0 | 1.4 | 100.0 | 0.0 |
| 5 | Run | 3.0 | 1.4 | 100.0 | 0.0 |
| 6 | Run | 1.0 | 1.4 | 7.0 | 93.0 |
| 6 | Washing | 4.0 | 1.4 | 7.0 | 93.0 |

Under the experimental conditions employed, using a PerkinElmer Flexar Module system with photodiode detector and Chromera™ analysis software, the retention times in minutes were 6.19 for neoeriocitrin, 7.81 for naringin, 9.02 for neohesperidin, 11.75 for melitidin and 13.21 for brutieridin.

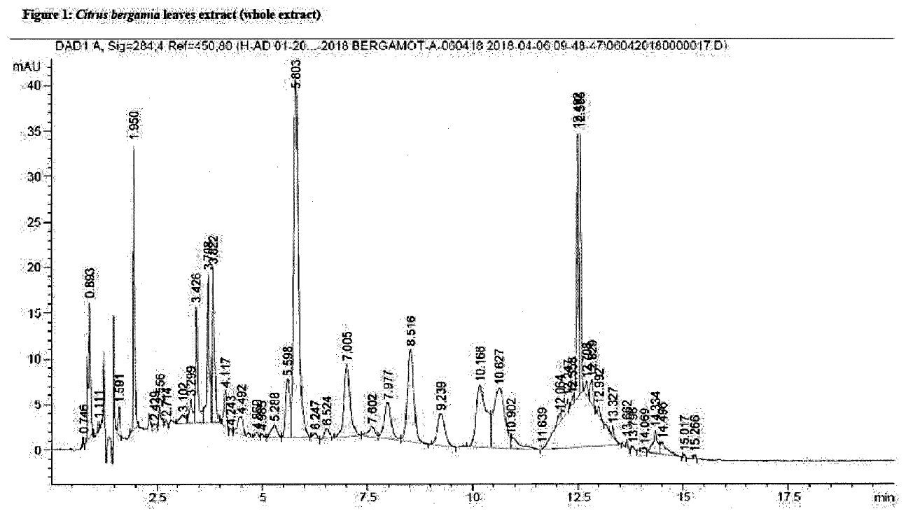

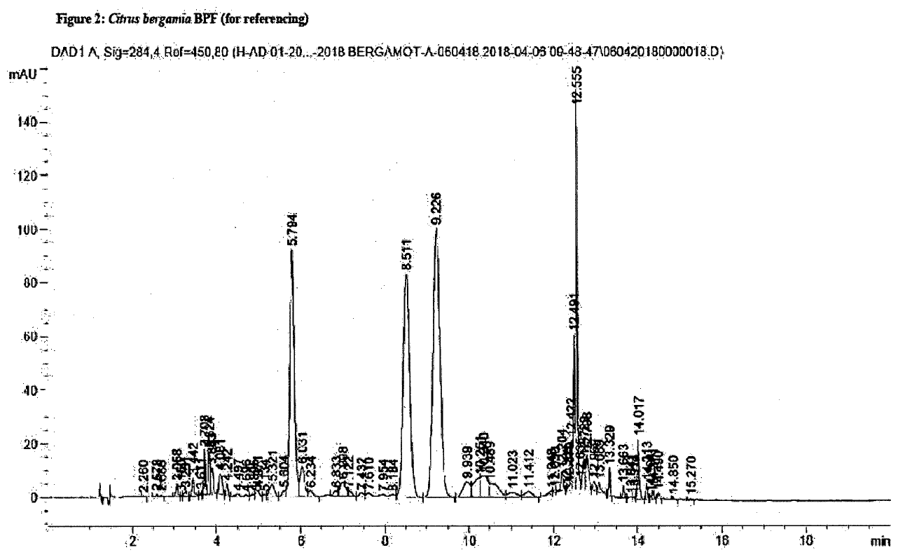

FIG. 1 shows the chromatogram of the novel extract obtained from Citrus aurantium var. bergamia leaves, and FIG. 2 shows the extract obtained from the fruit by the procedures reported in U.S. Pat. No. 8,741,362. The difference in composition of the two extracts is evident.

The extract is made by freezing, thawing and pressing the biomass, followed by purification systems as described in U.S. Pat. No. 8,741,362. The biomass consists of leaf masses of Citrus aurantium var. bergamia, and other varieties such as the troyan and femminello cultivars, selected for their active ingredient content. The original plant can be reproduced by micropropagation.

The leaf mass is extracted using water as the only extraction solvent.

The leaf biomass is pre-frozen by being passed through a liquid-air tunnel, wherein freezing takes place in a few seconds; the frozen leaf mass can be crushed by cryomilling and then heated to a temperature of between 2 and 10° C., preferably 5° C., and processed immediately or heated to temperatures ranging between 60 and 95°, preferably 75°. At said temperatures, inhibition of the oxidative and hydrolytic enzymes is reduced to acceptable limits, with a positive, economically valid energy balance.

The cryomilled biomass, acidified with 2N hydrochloric acid to pH 2.5 and brought to room temperature, is extracted by pressing at pressures ranging between 150 and 200 bars, with an aqueous counterwash and repressing. The aqueous extract undergoes centrifugation to eliminate undesirable insolubles and ultrafiltrate. In the case of heat shock at 75°, the biomass is directly pressed and counterwashed by the procedures described above. The crushing liquid, which is cloudy due to the presence of plant material and glycoside polymers, is centrifuged and ultrafiltered.

The clear extracts obtained after centrifugation and ultrafiltration are passed through SEPABEADS SP adsorption resin or other polystyrene resins; the resin, which retains the polyphenol substances, is washed thoroughly with water to remove inert substances, and the retentate is then eluted with a water-alcohol solution, preferably an alcohol solution with an 8:2 ethanol/water ratio, and the eluate is concentrated until dry in a vacuum. The dried extract takes the form of a beige powder.

Pure neoeriocitrin can be isolated from said extract by a chromatography process and crystallisation, as described in example 4.

The extract according to the invention has a particularly significant biological action on both lipid metabolism and blood sugar metabolism. The leaf extract according to the invention was compared with the fruit extract in an experimental model according to a crossed treatment design. Normal rats, Zucker fatty rats and Zucker diabetic fatty (ZDF) rats were treated for that purpose.

The results confirmed the advantageous activity of the extract. The extract according to the invention can be combined with other phytotherapeutic extracts, such as Cynara scolymus Cynara cardunculus, Cynara cardunculus var. sylvestris, Olea oleracea, Berberis aristata, Cyclanthera pedata, Gymnema sylvestre, Eugenia jambolana, Vitis vinifera and/or carotenoid extracts. Combinations with Cyclanthera pedata, Gymnema sylvestre and Eugenia jambolana extracts are particularly useful as antidiabetics, as they normalise the carbohydrate and lipid parameters of interest in the treatment of atherosclerotic syndromes.

The formulations according to the invention have also proved effective on different parameters in a range of patients suffering from metabolic syndrome, in whom normalisation of blood glucose, lipid parameters, hypertension and “silent inflammation” was observed.

According to a preferred aspect, the compositions according to the invention will be formulated as conventional or gastroprotected capsules or tablets, soft gelatin capsules or hard capsules with an oil content. The combination with oils rich in ω-3 fatty acids and phospholipids facilitates absorption of the polymeric flavonoids of the extract.

According to a further aspect, the compositions according to the invention may be administered together with other substances having a useful or complementary activity.

The compositions according to the invention will be formulated by conventional methods, such as those described in “Remington's Pharmaceutical Handbook”, Mack Publishing Co., N.Y., USA. In particular, the compositions according to the invention will be formulated according to conventional plant ingredient formulation techniques, which require particular care to be taken to avoid interactions with the excipients and the capsule matrices. Examples of oral formulations are tablets, drages, soft and hard gelatin capsules, and cellulose capsules.

The examples below further illustrate the invention.

### Example 1—Extraction

100 kg of fresh leaves of cultivated Citrus aurantium var. bergamia are rapidly frozen in a liquid-air tunnel to a temperature of −20 degrees, and immediately ground in a cryomill; the frozen powder is then conveyed through a second tunnel with steam heating before being introduced into an extractor under pressure. The biomass is pressed at 200 bars, separating the liquid and counterwashing the pressing with 2 volumes of demineralised water. The combined liquids are clarified by centrifugation and clarified by ultrafiltration, and absorbed on SEPABEADS SP resin to concentrate the polyphenol substances; the resin is washed thoroughly with water to remove inert substances, and then eluted with an 8:2 ethanol:water mixture. The water-alcohol solution is concentrated to recover the ethanol, and the aqueous solution is concentrated in a vacuum to a weight residue of 10%, and atomised. 0.82 Kg of a yellowish product with a total polyphenol content of 81%, expressed as neoeriocitrin with the Folin-Ciocalteu method described above, is obtained.

### Example 2—Extraction

100 kg of fresh leaves of cultivated Citrus aurantium var. bergamia are rapidly frozen in a liquid-air tunnel to a temperature of −20° C., and immediately ground in a cryomill; the frozen powder is acidified to pH 2.5 with 2N hydrochloric acid, and heated to ambient temperature (25-30° C.) before being introduced into the extractor under pressure. The biomass is pressed at 200 bars, separating the liquid and counterwashing the pressing with 2 volumes of demineralised water. The combined liquids are clarified by centrifugation and clarified by ultrafiltration, and absorbed on SEPABEADS SP resin to concentrate the polyphenol substances; the resin is washed thoroughly with water to remove inert substances, and then eluted with a 3:7 mixture of water:acetone. The water-acetone solution is concentrated to recover the acetone, and the residual aqueous solution is concentrated in a vacuum to a weight residue of 10%, and atomised. 0.71 Kg of a yellowish product with a total polyphenol content of 86%, expressed as neoeriocitrin with the Folin-Ciocalteu method, is obtained.

### Example 3—800 mg Soft Gelatin Capsules

Unit composition

| | |
|---|---|
| Extract of example 1 | 300 mg |
| Soya lecithin | 200 mg |
| Linseed oil | 300 mg |

### Example 4—Preparation of Pure Neoeriocitrin

100 g of the extract prepared according to example 1 is dissolved in 250 ml of ethanol and absorbed on silica gel in a column containing 2 kg of silica gel, and the product is eluted with a mixture of ethyl acetate:ethanol:water at the ratio of 100:13.5:10, collecting one-litre fractions and monitoring the fractions containing neoeriocitrin by TLC. Said fractions are concentrated until dry, and the residue is chromatographed by HPLC.

1. Extracts of the aerial parts of Citrus aurantium var. bergamia or Citrus aurantium var. myrtifolia.

2. Extracts according to claim 1 wherein the aerial parts are the leaves.

3. Extracts according to claim 1 of the leaves of the femminello and troyan cultivars of Citrus aurantium var. bergamia.

4. Extracts according to claim 1 characterised by a neoeriocitrin content 50 to 90% higher than that of the other flavonoids, an oligomeric procyanidin content ranging from 15 to 30% by weight of the extract, and the absence of furanocoumarins and coumarins.

5. Compositions comprising the extracts of claim 1, in admixture with suitable excipients, and optionally with other phytotherapeutic extracts, as active ingredients.

6. Compositions according to claim 5 wherein the phytotherapeutic extracts comprise extracts of Cynara scolymus, Cynara cardunculus, Cynara cardunculus var. sylvestris, Olea oleracea, Berberis aristata, Cyclanthera pedata, Gymnema sylvestre, Eugenia jambolana, Vitis vinifera and/or carotenoids.

7. Method of controlling lipid metabolism and of treating or preventing metabolic syndrome in patients in need thereof with the extracts according to claim 1, said method comprising
administering a pharmaceutical effective amount of said extracts to said patients in need thereof.
